(19) United States
(12) Patent Application Publication
(10) Pub. No.: US 2025/0339524 A1
(43) Pub. Date: Nov. 6, 2025

(54) COMBINATION ANTI-4-1BB IMMUNOTHERAPY, RADIOTHERAPY, AND B CELL ACTIVATION THERAPY

(71) Applicant: H. LEE MOFFITT CANCER CENTER AND RESEARCH INSTITUTE INC., Tampa, FL (US)

(72) Inventors: Bradford PEREZ, Tampa, FL (US); Jose CONEJO-GARCIA, Durham, NC (US)

(21) Appl. No.: 18/862,276

(22) PCT Filed: May 3, 2023
(86) PCT No.: PCT/US2023/066528

§ 371 Date: Nov. 1, 2024

Related U.S. Application Data
(60) Provisional application No. 63/364,079, filed on May 3, 2022.
(60) Provisional application No. 63/348,129, filed on Jun. 2, 2022.

(51) Int. Cl. A61K 39/395; A61K 31/711; A61K 38/20; A61K 38/21; A61K 39/00; A61N 5/10; A61P 35/00

(52) U.S. Cl. CPC A61K 39/39558; A61K 31/711; A61K 38/2013; A61K 38/204; A61K 38/2066; A61K 38/2086; A61K 38/212; A61P 35/00; A61K 2039/505; A61K 2039/545; A61N 5/10

(57) ABSTRACT

Disclosed herein is a method of treating a tumor in a subject that involves a) treating the tumor with an effective amount of radiotherapy: b) treating the subject with an effective amount of a B cell agonist: and c) administering to the subject a 4-1BB agonist. In some embodiments. the 4-1BB agonist is an isolated monoclonal antibody. or antigen binding portion thereof, that specifically binds human CD137 (4-1BB).

### CROSS-REFERENCE TO RELATED APPLICATIONS

This application claims benefit of U.S. Provisional Application No. 63/364,079, filed May 3, 2023, and U.S. Provisional Application No. 63/348,129, filed Jun. 2, 2022, which are hereby incorporated herein by reference in their entireties.

### STATEMENT REGARDING FEDERALLY SPONSORED RESEARCH OR DEVELOPMENT

This invention was made with Government Support under Grant No. CA231454 awarded by the National Institutes of Health. The Government has certain rights in the invention.

### SEQUENCE LISTING

This application contains a sequence listing filed in ST.26 format entitled “320803_2670_Sequence_Listing” created on May 2, 2023. The content of the sequence listing is incorporated herein in its entirety.

### BACKGROUND

Immunotherapy with checkpoint inhibitors targeting PD1, PD-L1 and CTLA4 have shown impressive gains compared to conventional chemotherapy for some patients with metastatic melanoma (Munhoz R R, et al. Cancer J. 2018 24(1):7-14; Larkin J, et al. JAMA Oncol. 2015 1(4):433-40; Hodi F S, et al. N Engl J Med. 2010 363(8):711-23) and lung cancer (Brahmer J, et al. N Engl J Med. 2015 373(2):123-35; Rizvi N A, et al. Lancet Oncol. 2015 16(3):257-65; Borghaei H, et al. The New England journal of medicine. 2015). Trials of TIGIT (Cancer Discov. 2020 10(8):1086-7) and other immune checkpoint inhibitors (Tawbi H A, et al. N Engl J Med. 2022 386(1):24-34) in combination with anti-PD1/PD-L1 immune checkpoint therapies are showing signs that novel combinations of checkpoint inhibitors may improve outcomes even further. Immune costimulatory agonist antibodies represent another class of immune therapies which have shown significant responses in murine models (Eskiocak U, et al. JCI Insight. 2020 5(5); Amatore F, et al. Expert Opin Biol Ther. 2020 20(2):141-50; Aspeslagh S, et al. Eur J Cancer. 2016 52:50-66) although early clinical trials are ongoing with mixed results. These drugs have been demonstrated to impact not only CD8 T cells but also CD4 T cells where they may play an important role in coordinating recruitment and proliferation of B cells and other antigen presenting cells as well as tertiary lymphoid structures and rich humoral responses. The mixed success of many agonist antibodies in early phase clinical trials may be due to poor agonist activity (Cohen E E W, et al. J Immunother Cancer. 2019 7(1):342) or off target effects/toxicities limiting opportunities for dose escalation (Segal N H, et al. Clin Cancer Res. 2017 23(8):1929-36). 2^{nd} generation co-stimulatory antibodies targeting 41BB may offer more appropriate activation without off target toxicities (Eskiocak U, et al. JCI Insight. 2020 5(5); Qi X, et al. Nat Commun. 2019 10(1):2141). Recently, many clinical trials have sought to combine radiation therapy (RT) with novel immune checkpoint therapies. These studies have met with mixed early results (McBride S, et al. J Clin Oncol. 2021; 39(1):30-7; Theelen W, et al. JAMA Oncol. 2019; Lee N Y, et al. Lancet Oncol. 2021 22(4):450-62; Tree A C, et al. Int J Radiat Oncol Biol Phys. 2018 101(5):1168-71) and although randomized comparisons to demonstrate synergy are ongoing (Jagodinsky J C, et al. Int J Radiat Oncol Biol Phys. 2020 108(1):6-16) (NCT03646617, NCT03811002, NCT04081688, NCT02444741), very little is understood about the nature of the RT associated immune response to help guide the design of next generation clinical trials.

### SUMMARY

Disclosed herein is a method of treating a tumor in a subject that involves a) treating the tumor with an effective amount of radiotherapy; b) treating the subject with an effective amount of a B cell agonist; and c) administering to the subject a 4-1BB agonist. In some embodiments, the 4-1BB agonist is an isolated monoclonal antibody, or antigen binding portion thereof, that specifically binds human CD137 (4-1BB).

In some embodiments, the method further involves administering to the patient an additional cancer treatment. In some embodiments, the additional cancer treatment is selected from the group comprising surgery, radiotherapy, chemotherapy, toxin therapy, immunotherapy, cryotherapy and gene therapy; optionally wherein said chemotherapy comprises (a) administering to the patient a drug selected from the group comprising oxaliplatin, doxorubicin, daunorubicin, docetaxel, mitoxanthrone, paclitaxel, digitoxin, digoxin, and septacidin and/or (b) administering to the patient a drug formulation selected from the group comprising a polymeric micelle formulation, a liposomal formulation, a dendrimer formulation, a polymer-based nanoparticle formulation, a silica-based nanoparticle formulation, a nanoscale coordination polymer formulation, a nanoscale metal-organic framework formulation, and an inorganic nanoparticle formulation.

In some embodiments, the method further involves treating the subject with a chemotherapeutic, immunotherapeutic, or combination thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show B cells and CD4 T Cells Migrate to the Tumor Microenvironment after Radiotherapy. FIG. 1A shows an experimental Design. 10\hat{ }6 B16-F10 melanoma cells transduced with OVA-antigen were injected subcutaneous bilateral flanks of C57/BL6 mice. Upon tumor growth around 10-12 days, treatment to the right flank was initiated with total dose of 8Gy×3 daily. Tumor microenvironment by flow cytometry at 5 and 10 days following radiotherapy. FIG. 1B, left shows CD4/CD8 ratio increases after RT on day 5, with a subsequent decrease on day 10. FIG. 1B, right shows % CD4 of CD45+ live cells increases in response to RT on day 5 with no significant changes on day 10. FIG. 1C shows % CD8 of CD45+ live cells demonstrates no significant change on day 5 with significantly increased % CD8 T cells 10 days after RT. FIG. 1D, Left shows % B cells (CD45+B220+) is significantly increased on day 5 with no significant change on day 10. FIG. 1D, Right shows % activated (CD86+) B cells are significantly increased on days 5 and 10 after radiation. FIG. 1E, Left shows % dendritic cells (CD11c+) in the CD45+ compartment have no significant change on day 5 or day 10 after RT. FIG. 1E, Right shows % activated (CD86+) dendritic cells are significantly increased on days 5 and 10 after radiation. RT, radiotherapy. CTRL, control. ns, not significant. DC, dendritic cell.

FIGS. 2A to 2D show CD4 T cells demonstrate increased evidence of antigen exposure at the tumor site and spleen in mice and in human peripheral blood with RT. FACS analysis demonstrating increased antigen exposed CD4+CD44+ T cells at the tumor site (FIG. 2A) and in the peripheral spleen (FIG. 2B) in B16-ova mouse model described in FIG. 1A. FIG. 2C is a schematic of experimental design of human small cell lung cancer clinical trial demonstrating peripheral blood collection at baseline and within 72 hrs after receipt of 30Gy thoracic RT in 10 fractions. FIG. 2D shows FACS analysis demonstrating similarly increased antigen-exposed CD4+CD45RA- memory T cells following RT in patients with small cell lung cancer receiving thoracic RT.

FIGS. 3A to 3H show radiation Activates T cells and B to Increase Expression of 4-1BB (CD137) in the B16-Ova murine model and in patients with ES-SCLC. FIG. 3A shows FACS analyses demonstrating statistically significant increase % 41BB+ CD4 cells among irradiated tumors at both 5- and 10-days following RT. FIG. 3B shows significant increase % 41BB+ B cells among irradiated tumors at 5- days but not 10-days following RT. FIG. 3C shows no difference in % 41BB+ CD8 T cells at 5 days after but significantly Increased % 41BB+ CD8 cells among irradiated tumors 10 days following RT. FIG. 3D shows FACS demonstrating low expression with no significant change in 41BB-expressing dendritic cells among irradiated tumors at either temporal point. FIG. 3E shows FACS analyses demonstrating increased % 41BB+ peripheral CD4 T cells in a patient with extensive stage small cell lung cancer receiving thoracic RT on clinical trial as described in FIG. 2C. FIG. 3F shows composite analysis demonstrating fold change increase in CD4+4-1BB+ above baseline after RT for almost all patients (left) and absolute change from baseline (right) of 4-1BB+ CD4 T cells following RT. FIG. 3G shows FACS demonstrating increased 4-1BB+, CD69+ CD4 T cells following RT in representative patient with ES-SCLC. FIG. 3H FACS composite analyses of all patients on SCLC trial with available data before and after RT demonstrating statistically significant increased fold change above baseline (left) and absolute change from baseline (right) of 4-1BB+, CD69+ CD4 T cells.

FIGS. 4A to 44K show the addition of 4-1BB to RT leads to more pronounced immune cell activation and migration to the tumor microenvironment B16-ova tumor bearing C57/B6 mice. Experimental Design. 10\hat{ }6 B16-F10 melanoma cells transduced with OVA-antigen were injected subcutaneous bilateral flanks of C57/BL6 mice. Upon tumor growth around 10-12 days, treatment to the right flank was initiated with total dose of 8Gy×3 daily with or without concurrent 100 ug 4-1BB antibody (mAb22). Mice were euthanized and tumors were evaluated 10 days following treatment. Tumor microenvironment by FACS at 10 days following radiotherapy. FIG. 4A shows CD4/CD8 ratio after RT decreases significantly with 4-1BB antibody (mAb22) in combination with RT. FIG. 4B shows % CD8 T cells in the CD45 compartment increases significantly with 4-1BB therapy, and even more so with the combination therapy, without significant change in the proportion of CD4 cells (FIG. 4C). B cells (B220+) show increased % CD80 (FIG. 4D) and % CD86+ cells (FIG. 4E) + with 4-1BB therapy, and are increased even further with combination 4-1BB and RT. Similar increases in CD80%+ (FIG. 4F) and CD86%+ (FIG. 4G) are seen among dendritic cells treated with 41BB and 41BB+ XRT treatments. FIG. 4H shows no significant changes in the CD4/CD8 ratio in either of the bilateral draining lymph nodes 10 days after RT. FIG. 41 shows increased expression of CD40L on CD4 T cells in bilateral draining lymph nodes in response to combination 41BB and RT, without significant changes with 41BB alone compared to no treatment control. FIG. 4J shows no significant changes in proportion of dendritic cells in CD45 compartment in bilateral draining lymph nodes in response to either 4-1BB alone or the combination of 4-1BB and RT. FIG. 4K shows the proportion of dendritic cells (CD11c+) expressing CD103 in bilateral draining lymph nodes is significantly increased with combination 4-1BB and RT without significant changes with 4-1BB alone, compared to no treatment controls.

FIGS. 5A to 5F show 4-1BB and RT-induced anti-tumor immune activation improves tumor control at unirradiated tumor sites in 2 unique tumor models. 10\hat{ }6 B16 -F10 melanoma cells transduced with OVA-antigen were injected subcutaneous bilateral flanks of C57/BL6 mice. Upon tumor growth around 10-12 days, treatment to the right flank was initiated with total dose of 8Gy×3 daily with or without concurrent 100 ug 4-1BB antibody (mAb22). Mice were followed until euthanasia criteria were met. FIGS. 5A to 5D show tumor growth curves (Right) and Kaplan Meier time to contralateral flank growth of 150 mm3 (Left). FIG. 5A shows tumor growth in left flank of untreated mice inoculated with 10\hat{ }6 B16-ova. FIG. 5B shows tumor growth of unirradiated left flanks among mice receiving XRT alone to the right flank compared to no treatment. FIG. 5C shows tumor growth in unirradiated left flanks among mice receiving XRT to right flank with concurrent 4-1BB antibody (mAb22) compared to no treatment. 10\hat{ }6 Lewis lung carcinoma (LLC) cells were injected into subcutaneous bilateral flanks of C57/B6 mice and mice were similarly treated with radiation to the right flank to a total dose of 8Gy×3 daily with or without concurrent 100 ug 4-1BB antibody (3H3). Inoculation of the left flank was delayed by 12 days. Tumor growth curves (Right) and Kaplan Meier time to unirradiated flank growth of 150 mm³ (Left). FIG. 5D shows tumor growth in left flank of untreated mice inoculated with 10\hat{ }6 LLC. FIG. 5E shows tumor growth of unirradiated left flanks among mice receiving XRT alone to the right flank compared to no treatment. FIG. 5F shows tumor growth in unirradiated left flanks among mice receiving XRT to right flank with concurrent 4-1BB antibody (mAb22) compared to no treatment.

FIGS. 6A to 6D show 4-1BB and RT-induced tumor control at unirradiated tumor sites is dependent on infiltration of B cells, as well as CD4 and CD8 T cells. FIG. 6A shows B16-Ova tumor growth in unirradiated left flanks among mice receiving XRT to right flank with concurrent 4-1BB antibody (mAb22). FIG. 6B shows addition of B cell depleting antibody (anti-B220, 300 ug) two days following initiation of XRT compared to similarly treated cohort without B cell depletion. FIG. 6C show tumor growth in unirradiated left flanks among mice receiving XRT to right flank with concurrent 4-1BB antibody (mAb22) and CD4 depleting antibody (anti-CD4, 200 ug) two days following initiation of XRT compared to similarly treated cohort without CD4 T cell depletion. FIG. 6D shows tumor growth in unirradiated left flanks among mice receiving XRT to right flank with concurrent 4-1BB antibody (mAb22) and CD8 depleting antibody (anti-CD8, 200 ug) two days following initiation of XRT compared to similarly treated cohort without CD8 T cell depletion.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Disclosed herein is a method of treating a tumor in a subject that involves a) treating the tumor with an effective amount of radiotherapy; b) treating the subject with an effective amount of a B cell agonist; and c) administering to the subject an isolated monoclonal antibody, or antigen binding portion thereof, that specifically binds human CD137 (4-1BB).

### Radiotherapy

Radiation therapy is the medical use of high-energy radiation (e.g., x-rays, gamma rays, charged particles) to shrink tumors and kill malignant cells, and is generally used as part of cancer treatment. Radiation therapy kills malignant cells by damaging their DNA.

Radiation therapy can be delivered to a patient in several ways. For example, radiation can be delivered from an external source, such as a machine outside the patient's body, as in external beam radiation therapy. External beam radiation therapy for the treatment of cancer uses a radiation source that is external to the patient, typically either a radioisotope, such as 60Co, 137Cs, or a high energy x-ray source, such as a linear accelerator. The external source produces a collimated beam directed into the patient to the tumor site. External-source radiation therapy avoids some of the problems of internal-source radiation therapy, but it undesirably and necessarily irradiates a significant volume of non-tumorous or healthy tissue in the path of the radiation beam along with the tumorous tissue.

The adverse effect of irradiating of healthy tissue can be reduced, while maintaining a given dose of radiation in the tumorous tissue, by projecting the external radiation beam into the patient at a variety of “gantry” angles with the beams converging on the tumor site. The particular volume elements of healthy tissue, along the path of the radiation beam, change, reducing the total dose to each such element of healthy tissue during the entire treatment.

The irradiation of healthy tissue also can be reduced by tightly collimating the radiation beam to the general cross section of the tumor taken perpendicular to the axis of the radiation beam. Numerous systems exist for producing such a circumferential collimation, some of which use multiple sliding shutters which, piecewise, can generate a radio-opaque mask of arbitrary outline.

For administration of external beam radiation, the amount can be at least about 1 Gray (Gy) fractions at least once every other day to a treatment volume. In a particular embodiment, the radiation is administered in at least about 2 Gray (Gy) fractions at least once per day to a treatment volume. In another particular embodiment, the radiation is administered in at least about 2 Gray (Gy) fractions at least once per day to a treatment volume for five consecutive days per week. In another particular embodiment, radiation is administered in 10 Gy fractions every other day, three times per week to a treatment volume. In another particular embodiment, a total of at least about 20 Gy is administered to a patient in need thereof. In another particular embodiment, at least about 30 Gy is administered to a patient in need thereof. In another particular embodiment, at least about 40 Gy is administered to a patient in need thereof.

Typically, the patient receives external beam therapy four or five times a week. An entire course of treatment usually lasts from one to seven weeks depending on the type of cancer and the goal of treatment. For example, a patient can receive a dose of 2 Gy/day over 30 days.

Internal radiation therapy is localized radiation therapy, meaning the radiation source is placed at the site of the tumor or affected area. Internal radiation therapy can be delivered by placing a radiation source inside or next to the area requiring treatment. Internal radiation therapy is also called brachytherapy. Brachytherapy includes intercavitary treatment and interstitial treatment. In intracavitary treatment, containers that hold radioactive sources are put in or near the tumor. The sources are put into the body cavities. In interstitial treatment, the radioactive sources alone are put into the tumor. These radioactive sources can stay in the patient permanently. Typically, the radioactive sources are removed from the patient after several days. The radioactive sources are in containers.

There are a number of methods for administration of a radiopharmaceutical agent. For example, the radiopharmaceutical agent can be administered by targeted delivery or by systemic delivery of targeted radioactive conjugates, such as a radiolabeled antibody, a radiolabeled peptide and a liposome delivery system. In one particular embodiment of targeted delivery, the radiolabelled pharmaceutical agent can be a radiolabelled antibody. See, for example, Ballangrud A. M., et al. Cancer Res., 2001; 61:2008-2014 and Goldenber, D. M. J. Nucl. Med., 2002; 43 (5):693-713, the contents of which are incorporated by reference herein.

In another particular embodiment of targeted delivery, the radiopharmaceutical agent can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. See, for example, Emfietzoglou D, Kostarelos K, Sgouros G. An analytical dosimetry study for the use of radionuclide-liposome conjugates in internal radiotherapy. J Nucl Med 2001; 42:499-504, the contents of which are incorporated by reference herein.

In yet another particular embodiment of targeted delivery, the radiolabeled pharmaceutical agent can be a radiolabeled peptide. See, for example, Weiner R E, Thakur M L. Radiolabeled peptides in the diagnosis and therapy of oncological diseases. Appl Radiat Isot 2002 November; 57(5):749-63, the contents of which are incorporated by reference herein.

In addition to targeted delivery, brachytherapy can be used to deliver the radiopharmaceutical agent to the target site. Brachytherapy is a technique that puts the radiation sources as close as possible to the tumor site. Often the source is inserted directly into the tumor. The radioactive sources can be in the form of wires, seeds or rods. Generally, cesium, iridium or iodine are used.

Systemic radiation therapy is another type of radiation therapy and involves the use of radioactive substances in the blood. Systemic radiation therapy is a form of targeted therapy. In systemic radiation therapy, a patient typically ingests or receives an injection of a radioactive substance, such as radioactive iodine or a radioactive substance bound to a monoclonal antibody.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

As used herein, a “metallic radioisotope” is any suitable metallic radioisotope useful in a therapeutic or diagnostic procedure in vivo or in vitro. Suitable metallic radioisotopes include, but are not limited to: Actinium-225, Antimony-124, Antimony-125, Arsenic-74, Barium-103, Barium-140, Beryllium-7, Bismuth-206, Bismuth-207, Bismuth212, Bismuth213, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-60, Copper-62, Copper-64, Copper-67, Erbium-169, Europium-152, Gallium-64, Gallium-67, Gallium-68, Gadolinium153, Gadolinium-157 Gold-195, Gold-199, Hafnium-175, Hafnium-175-181, Holmium-166, Indium-110, Indium-111, Iridium-192, Iron 55, Iron-59, Krypton85, Lead-203, Lead-210, Lutetium-177, Manganese-54, Mercury-197, Mercury203, Molybdenum-99, Neodymium-147, Neptunium-237, Nickel-63, Niobium95, Osmium-185+191, Palladium-103, Palladium-109, Platinum-195m, Praseodymium-143, Promethium-147, Promethium-149, Protactinium-233, Radium-226, Rhenium-186, Rhenium-188, Rubidium-86, Ruthenium-97, Ruthenium-103, Ruthenium-105, Ruthenium-106, Samarium-153, Scandium-44, Scandium-46, Scandium-47, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, Zirconium-89, and Zirconium-95.

As used herein, a “non-metallic radioisotope” is any suitable nonmetallic radioisotope (non-metallic radioisotope) useful in a therapeutic or diagnostic procedure in vivo or in vitro. Suitable non-metallic radioisotopes include, but are not limited to: Iodine-131, lodine-125, lodine-123, Phosphorus-32, Astatine-211, Fluorine-18, Carbon-11, Oxygen-15, Bromine-76, and Nitrogen-13.

Identifying the most appropriate isotope for radiotherapy requires weighing a variety of factors. These include tumor uptake and retention, blood clearance, rate of radiation delivery, half-life and specific activity of the radioisotope, and the feasibility of large-scale production of the radioisotope in an economical fashion. The key point for a therapeutic radiopharmaceutical is to deliver the requisite amount of radiation dose to the tumor cells and to achieve a cytotoxic or tumoricidal effect while not causing unmanageable side-effects.

It is preferred that the physical half-life of the therapeutic radioisotope be similar to the biological half-life of the radiopharmaceutical at the tumor site. For example, if the half-life of the radioisotope is too short, much of the decay will have occurred before the radiopharmaceutical has reached maximum target/background ratio. On the other hand, too long a half-life could cause unnecessary radiation dose to normal tissues. Ideally, the radioisotope should have a long enough half-life to attain a minimum dose rate and to irradiate all the cells during the most radiation sensitive phases of the cell cycle. In addition, the half-life of a radioisotope has to be long enough to allow adequate time for manufacturing, release, and transportation.

Other practical considerations in selecting a radioisotope for a given application in tumor therapy are availability and quality. The purity has to be sufficient and reproducible, as trace amounts of impurities can affect the radiolabeling and radiochemical purity of the radiopharmaceutical.

The target receptor sites in tumors are typically limited in number. As such, it is preferred that the radioisotope have high specific activity. The specific activity depends primarily on the production method. Trace metal contaminants must be minimized as they often compete with the radioisotope for the chelator and their metal complexes compete for receptor binding with the radiolabeled chelated agent.

The type of radiation that is suitable for use in the methods of the present invention can vary. For example, radiation can be electromagnetic or particulate in nature. Electromagnetic radiation useful in the practice of this invention includes, but is not limited to, x-rays and gamma rays. Particulate radiation useful in the practice of this invention includes, but is not limited to, electron beams (beta particles), protons beams, neutron beams, alpha particles, and negative pi mesons. The radiation can be delivered using conventional radiological treatment apparatus and methods, and by intraoperative and stereotactic methods. Additional discussion regarding radiation treatments suitable for use in the practice of this invention can be found throughout Steven A. Leibel et al., Textbook of Radiation Oncology (1998) (publ. W. B. Saunders Company), and particularly in Chapters 13 and 14. Radiation can also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods can be used in the practice of this invention.

For tumor therapy, both at and B-particle emitters have been investigated. Alpha particles are particularly good cytotoxic agents because they dissipate a large amount of energy within one or two cell diameters. The β-particle emitters have relatively long penetration range (2-12 mm in the tissue) depending on the energy level. The long-range penetration is particularly important for solid tumors that have heterogeneous blood flow and/or receptor expression. The β-particle emitters yield a more homogeneous dose distribution even when they are heterogeneously distributed within the target tissue.

### B-Cell Activators

Various B-cell activators can be used in the methods and systems disclosed herein. Examples of B-cell activators include, but are not limited to, CpG DNA, IL-2, IL-4, IL-5, IL-6, IL-10, IL-15, IL-21, BAFF/BlyS, TACI, IFN-α, soluble CD40L, an intratumoral oncolytic virus expressing CD40L, and agonist anti-CD40.

### 4-1BB Agonist

### 4-1BB Ligand

The 4-1BB glycoprotein is a member of the tumor necrosis factor receptor superfamily and binds to a high-affinity ligand (4-1BBL) expressed on several antigen-presenting cells such as macrophages and activated B cells. Therefore, in some embodiments, the 4-1BB agonist is a 4-1BBL protein, or a fragment or variant thereof capable of ligating 4-1BB on T-cell. In some embodiments, the 4-1BB agonist is a recombinant protein, such as recombinant human 4-1BB. The recombinant protein can have the native 4-1BBL sequence. An example protein sequence for human 4-1BBL is provided in UniProtKB/Swiss-Prot Accession No. P41273. However, the recombinant protein can also be a fragment, variant, or combination thereof so long as it is capable of ligating 4-1BB on T-cell.

### Antibodies

In some embodiments, the 4-1BB agonist is an agonistic anti-4-1BB antibody capable of ligating 4-1BB on T-cells to induce its co-stimulatory activity. Suitable antibodies include both polyclonal and monoclonal antibodies. Antibodies that can be used in the disclosed compositions and methods include whole immunoglobulin (i.e., an intact antibody) of any class, fragments thereof, and synthetic proteins containing at least the antigen binding variable domain of an antibody.

The disclosed antibody can be a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

There are five major classes of human immunoglobulins: IgA, IgD, IgE, IgG and IgM, and several of these may be further divided into subclasses (isotypes), e.g., IgG-1, lgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively. The disclosed antibody can be of any of these classes so long as it is able to ligate 4-1BB on T-cells.

Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hyperyariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

The antibodies can also be “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

In vitro methods are also suitable for preparing monovalent antibodies. Digestion of antibodies to produce fragments thereof, particularly, Fab or F(ab)2 fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields an Fc fragment and an F(ab)2 fragment that has two antigen combining sites and is still capable of cross-linking antigen.

In some embodiments, the 4-1BB agonist is one of the agonistic antibodies described in, for example, U.S. Pat. Nos. 10,279,038 or 10,434,175, and U.S. Publication No. 2021/0221902, each of which are incorporated by reference in their entireties. For example, in some embodiments, the agonistic antibody comprises heavy and light chain CDRs selected from the group consisting of:
(a) heavy chain CDR1, CDR2 and CDR3 sequences set forth in FTFSSYAMS (SEQ ID NO: 1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLOS (SEQ ID NO:5), and QQGHLFPITF (SEQ ID NO:6), respectively (mAb1);
(b) heavy chain CDR1, CDR2 and CDR3 sequences set forth in FTFSSYAMS (SEQ ID NO:1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQNIHNWLAW (SEQ ID NO:7), YKASGLES (SEQ ID NO:8), and QQGDRFPLTF (SEQ ID NO:9), respectively (mAb2);
(c) heavy chain CDR1, CDR2 and CDR3 sequences set forth in FTFSSYAMS (SEQ ID NO:1), SAISGSGGSTYY (SEQ ID NO:2), and AKDSPFLLDDYYYYYY (SEQ ID NO: 3), respectively, and light chain CDR1, CDR2 and CDR3 sequences set forth in RASQSISRWLAW (SEQ ID NO: 10), FKASALES (SEQ ID NO:11), and QQGNSFPLTF (SEQ ID NO: 12), respectively (mAb3);
(d) heavy chain CDR1, CDR2 and CDR3 sequences set forth in FTFSSYAMS (SEQ ID NO:1), SAISGSGGSTYY (SEQ ID NO:2), and AKDSPFLLDDYYYYYYMB (SEQ ID NO:3), respectively, and light chain CDR1, CDR2 and CDR3 sequences set forth in RASQNIDIWLAW (SEQ ID NO: 13), YKASGLET (SEQ ID NO:14), and QQGNQFPLTF (SEQ ID NO:15), respectively (mAb4);
(e) heavy chain CDR1, CDR2 and CDR3 sequences set forth in FTFSSYAMS (SEQ ID NO: 1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQSIGRWLAW (SEQ ID NO: 16), FKASALEV (SEQ ID NO:17), and QQGNSFPLTF (SEQ ID NO:12), respectively (mAb5);
(f) heavy chain CDR1, CDR2 and CDR3 sequences set forth in FTFSSYAMS (SEQ ID NO:1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQSISSWLAW (SEQ ID NO:18), YAASALQS (SEQ ID NO:19), and QQGDSFPLTF (SEQ ID NO:20), respectively (mAb6);
(g) heavy chain CDR1, CDR2 and CDR3 sequences set forth in FTFSSYAMS (SEQ ID NO: 1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQSINTWLAW (SEQ ID NO:21), YKASALEN (SEQ ID NO:22), and QQGNSFPLTF (SEQ ID NO:12), respectively (mAb7);
(h) heavy chain CDR1, CDR2 and CDR3 sequences set forth in FTFSSYAMS (SEQ ID NO: 1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQSISSWLAW (SEQ ID NO: 18), YKASALES (SEQ ID NO:23), and QQGHSFPLTF (SEQ ID NO:24), respectively (mAb8);
(i) heavy chain CDR1, CDR2 and CDR3 sequences set forth FTFSSYAMS (SEQ ID NO: 1), SAISGSGGSTYY (SEQ ID NO:2), and AKDSPFLLDDYYYYYYMD (SEQ ID NO: 3), respectively, and light chain CDR1, CDR2 and CDR3 sequences set forth in RASQSISDWLAW (SEQ ID NO:25), FKASALEG (SEQ ID NO:26), and QQGNSFPITF (SEQ ID NO:27), respectively (mAb9);
(j) heavy chain CDR1, CDR2 and CDR3 sequences set forth in FTFSSYAMS (SEQ ID NO: 1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQSVDRWLAW (SEQ ID NO:28), YEASALQG (SEQ ID NO:29), and QQGDSFPLTF (SEQ ID NO:30), respectively (mAb10);
(k) heavy chain CDR1, CDR2 and CDR3 sequences set forth in FTFSSYAMS (SEQ ID NO:1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQGISSWLAW (SEQ ID NO:4), YAASGLQN (SEQ ID NO:31), and QQGDRFPLTF (SEQ ID NO:9), respectively (mAb11);
(l) heavy chain CDR1, CDR2 and CDR3 sequences set forth in FTFNYYAMS (SEQ ID NO:32), SAIDGSGDNTTY (SEQ ID NO:33), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb12);
(m) heavy chain CDR1, CDR2 and CDR3 sequences set forth in FTFNYYAMS (SEQ ID NO:32), AAISGSGDGTYY (SEQ ID NO:34), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb13);
(n) heavy chain CDR1, CDR2 and CDR3 sequences set forth in FTFNYYAMS (SEQ ID NO:32), SAISGSGDSTYY (SEQ ID NO:47), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb14);
(o) heavy chain CDR1, CDR2 and CDR3 sequences set forth in FTFNYYAMS (SEQ ID NO:32), AAISGGGDATYY (SEQ ID NO:35), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLOS (SEQ ID NO:5), and QQGHLFPITF (SEQ ID NO:6), respectively (mAb15);
(p) heavy chain CDR1, CDR2 and CDR3 sequences set forth in FTFYGYAMS (SEQ ID NO:36), SSISGSGDVTYY (SEQ ID NO:37), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb16);
(q) heavy chain CDR1, CDR2 and CDR3 sequences set forth in FTFYGYAMS (SEQ ID NO:36), AAISGSGDGTYY (SEQ ID NO:34), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLOS (SEQ ID NO:5), and QQGHLFPITF (SEQ ID NO:6), respectively (mAb17);
(r) heavy chain CDR1, CDR2 and CDR3 sequences set forth in FTFRNYAMS (SEQ ID NO:38), SAISGFGESTYY (SEQ ID NO:39), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb18);
(s) heavy chain CDR1, CDR2 and CDR3 sequences set forth in FTFNYYAMN (SEQ ID NO:40), AAISGSGGRTYY (SEQ ID NO:41), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLOS (SEQ ID NO:5), and QQGHLFPITF (SEQ ID NO:6), respectively (mAb19);
(t) heavy chain CDR1, CDR2 and CDR3 sequences set forth in FTFYGYAMS (SEQ ID NO:36), SAISGSGGNTSY (SEQ ID NO:48), and AKDSPFLLDDYYYYYYMD (SEQ ID NO:3), respectively, and light chain CDR1, CDR2 and CDR3 sequences set forth in SEQ ID NOs: RASQGISSWLAW (SEQ ID NO:4), YAASSLOS (SEQ ID NO:5), and QQGHLFPITF (SEQ ID NO:6), respectively (mAb20);
(u) heavy chain CDR1, CDR2 and CDR3 sequences set forth in FTFYGYAMS (SEQ ID NO:36), AAISGSGDSTYY (SEQ ID NO:42), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb21);
(v) heavy chain CDR1, CDR2 and CDR3 sequences set forth in FTFRNYAMS (SEQ ID NO:38), SAISGSGDTTYY (SEQ ID NO:43), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5), and QQGHLFPITF (SEQ ID NO:6), respectively (mAb22);
(w) heavy chain CDR1, CDR2 and CDR3 sequences set forth in FTFGWYAMS (SEQ ID NO:44), SAISGSGGSTYY (SEQ ID NO:2) and AKDSPFLLDDYYYYYYMD (SEQ ID NO:3), respectively, and light chain CDR1, CDR2 and CDR3 sequences set forth in RASQGISSWLAW (SEQ ID NO:4), YAASSLQS (SEQ ID NO:5) and QQGHLFPITF (SEQ ID NO:6), respectively (mAb23);
(x) heavy chain CDR1, CDR2 and CDR3 sequences set forth in FTFSSYAMS (SEQ ID NO: 1), SAISGSGGSTYY (SEQ ID NO:2), and AKDSPFLLDDYYYYYYMD (SEQ ID NO:3), respectively, and light chain CDR1, CDR2 and CDR3 sequences set forth in RASQDIGDWLAW (SEQ ID NO:45), YKASGLQS (SEQ ID NO:46), and QQGNQFPLTF (SEQ ID NO: 15), respectively (mAb24); and
(y) heavy chain CDR1, CDR2 and CDR3 sequences set forth in FTFYGYAMS (SEQ ID NO:36), SAISGSGDTTYY (SEQ ID NO:43), and AKDSPFLLDDYYYYYYMD (SEQ ID NO:3), respectively, and light chain CDR1, CDR2 and CDR3 sequences set forth in RASQGISSWLAW (SEQ ID NO:4), YAASSLOS (SEQ ID NO: 5), and QQGHLFPITF (SEQ ID NO:6), respectively (mAb25).

### Aptamers

In some embodiments, the 4-1BB agonist is an agonistic aptamer capable of ligating 4-1BB on T-cells to induce its co-stimulatory activity. The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

Nucleic acid aptamers are typically oligonucleotides ranging from 15-50 bases in length that fold into defined secondary and tertiary structures, such as stem-loops or G-quartets. Nucleic acid aptamers preferably bind the target molecule with a Kd less than 10-6, 10-8, 10-10, or 10-12. Nucleic acid aptamers can also bind the target molecule with a very high degree of specificity.

Nucleic acid aptamers are typically isolated from complex libraries of synthetic oligonucleotides by an iterative process of adsorption, recovery and re-amplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. Specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer.

Peptide aptamers are proteins that are designed to interfere with other protein interactions inside cells. They consist of a variable peptide loop attached at both ends to a scaffold. This double structural constraint greatly increases the binding affinity of the peptide aptamer to levels comparable to an antibody. The variable loop length is typically composed of about ten to twenty amino acids, and the scaffold may be any protein which has good solubility. Currently, the bacterial protein Thioredoxin-A is the most used scaffold protein, the variable loop being inserted within the reducing active site, the two Cysteines lateral chains being able to form a disulfide bridge. Peptide aptamer selection can be made using different systems, but the most used is currently the yeast two-hybrid system. Peptide aptamer can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### Chemotherapeutic

In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

Non-limiting examples of known cancer drugs includes Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate

Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Breyanzi (Lisocabtagene Maraleucel), Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPOX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Danyelza (Naxitamab-gqgk), Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-Izsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fam-Trastuzumab Deruxtecan-nxki, Fareston (Toremifene), Farydak (Panobinostat Lactate), Faslodex (Fulvestrant), FEC, Fedratinib Hydrochloride, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, Fulphila (Pegfilgrastim), FU-LV, Fulvestrant, Gamifant (Emapalumab-Izsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin

Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, Ixempra (Ixabepilone), Jakafi (Ruxolitinib Phosphate), JEB, Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lisocabtagene Maraleucel, Lomustine, Lonsurf (Trifluridine and Tipiracil Hydrochloride), Lorbrena (Lorlatinib), Lorlatinib, Lumoxiti (Moxetumomab Pasudotox-tdfk), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Margenza (Margetuximab-cmkb), Margetuximab-cmkb, Marqibo (Vincristine Sulfate Liposome), Matulane (Procarbazine Hydrochloride), Mechlorethamine Hydrochloride, Megestrol Acetate, Mekinist (Trametinib Dimethyl Sulfoxide), Mektovi (Binimetinib), Melphalan, Melphalan Hydrochloride, Mercaptopurine, Mesnex (Mesna), Methotrexate Sodium, Methylnaltrexone Bromide, Midostaurin, Mitomycin, Mitoxantrone Hydrochloride, Mogamulizumab-kpkc, Monjuvi (Tafasitamab-cxix), Moxetumomab Pasudotox-tdfk, Mozobil (Plerixafor), MVAC, Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Naxitamab-gqgk, Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Nyvepria (Pegfilgrastim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Orgovyx (Relugolix), Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat Lactate, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Relugolix, R-EPOCH, Retacrit (Epoetin Alfa), Retevmo (Selpercatinib), Revlimid (Lenalidomide), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib,

Selumetinib Sulfate, Siltuximab, Sipuleucel-T, Soltamox (Tamoxifen Citrate), Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomide, Temsirolimus, Tepadina (Thiotepa), Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib Dimethyl Sulfoxide, Trastuzumab, Trastuzumab and Hyaluronidase-oysk, Treanda (Bendamustine Hydrochloride), Trexall (Methotrexate Sodium), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ukoniq (Umbralisib Tosylate), Ultomiris (Ravulizumab-cwvz), Umbralisib Tosylate, Undencyca (Pegfilgrastim), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VelP, Velcade (Bortezomib), Vemurafenib, Venclexta (Venetoclax),

Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), and Zytiga (Abiraterone Acetate).

### Immunotherapy Agent

In some embodiments, the cancer therapeutic is a cancer immunotherapy agent. Immunotherapy refers to a treatment that uses a subject's immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. Immunotherapy may be administered via injection (e.g., intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In certain embodiments, immune checkpoint inhibitors can be an inhibitory nucleic acid molecule (e.g., an siRNA molecule, an shRNA molecule or an antisense RNA molecule) that inhibits expression of an immune checkpoint protein that inhibits expression of an immune checkpoint protein.

In some embodiments, the immunotherapy agent is selected from the group comprising an anti-CD52 antibody, an anti-CD20 antibody, an anti-CD20 antibody, anti-CD47 antibody an anti-GD2 antibody, a radiolabeled antibody, an antibody-drug conjugate, a cytokine, polysaccharide K and a neoantigen; optionally wherein said cytokine is an interferon, an interleukin, or tumor necrosis factor alpha (TNF-α), further optionally where said cytokine is selected from the group comprising IFN-α, INF-γ, IL-2, IL-12 and TNF-α. In some embodiments, the immunotherapy agent is selected from the group comprising Alemtuzumab, Ofatumumab, Rituximab, and the immunotherapy agents sold under the tradenames ZEVALIN™, ADCETRIST, KADCYLA™, and ONTAK™. In some embodiments, the immunotherapy agent is selected from the group comprising a PD-1 inhibitor, a PD-L1 inhibitor, a CTLA-4 inhibitor, an IDO inhibitor, and a CCR7 inhibitor.

### Cancers

In some embodiments, the methods described herein relate to the treatment of cancer. Examples of cancers that may treated by methods described herein include, but are not limited to, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, colorectal cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Anti-4-1BB Immunotherapy Works Synergistically With Radiotherapy to Induce B and T Cell Dependent Anti-Tumor Immune Activation and Improve Tumor Control at Unirradiated Sites.

One major benefit of RT is the localized nature of treatment which allows for targeting of tumor microenvironments while limiting systemic effects. RT has been previously reported as an immune stimulator (Vaes R D W, et al. Cells. 2021 10(4)). and has been shown to enhance the diversity of the T cell receptor repertoire and augment the T cell response (Twyman-Saint Victor C, et al. Nature. 2015 520(7547):373-7). In preclinical studies, RT has been shown to mediate tumor regression via adaptor protein stimulator of interferon genes (STING)-mediated cytosolic DNA sensing, with associated Type I interferon and adaptive immune responses (Deng L, et al. Immunity. 2014 41(5):843-52). Unfortunately, activation of this same STING/interferon pathway also drives myeloid derived suppressor cell (MDSC) mobilization and associated RT resistance (Liang H, et al. Nat Commun. 2017 8(1):1736). Studies to evaluate RT with novel costimulatory antibodies have shown evidence of synergy in other preclinical studies (Pilones KA, et al. Cancer Immunol Res. 2020; 8(8):1054-63; Niknam S, et al. Clin Cancer Res. 2018 24(22):5735-43). Previous work has demonstrated that RT+ anti-4-1BB combinations are synergistic and demonstrated improved anti-tumor immune response (Belcaid Z, et al. PLOS One. 2014 9 (7):e101764; Rodriguez-Ruiz M E, et al. Mol Cancer Ther. 2019 18(3):621-31; Shiao J C, et al. Adv Radiat Oncol. 2017 2(3):398-402).

As is the case with most of the cancer immunotherapy field, much of the focus with RT combination studies has been on CD8 T cells which are demonstrated to play a critical role in effector anti-tumor function (Lee Y, et al. Blood. 2009 114(3):589-95; Arina A, et al. Nat Commun. 2019 10(1):3959). CD8 T cells play a critical role and waves of renewable effector CD8 T cells are likely responsible for successful immune response against cancer (Eberhardt C S, et al. Nature. 2021 597(7875):279-84). What is not clear however, is how these renewable T cells are maintained and what is the makeup of the other immune cells which refresh and support effector CD8 T response.

It is known from decades of basic immunology (Whitmire J K, et al. J Immunol. 2009 182(4):1868-76; Murphy K, et al. Janeway's immunobiology. 8th ed. New York: Garland Science; 2012. xix, 868) and more recent attention in cancer immunology that CD4 T cells (Herrera FG, et al. Cancer Discov. 2022 12(1):108-33) and critically B cells (Franiak-Pietryga I, et al. Int J Radiat Oncol Biol Phys. 2022 112(2):514-28; Kim S S, et al. Clin Cancer Res. 2020 26(13):3345-59) are also important for mounting robust anti-tumor immune response following radiotherapy. In this study, an early influx of CD4 T cells and B cells were identified following flank radiotherapy in a murine model. As disclosed herein, these cells are required, along with CD8 T cells, for RT response with immunotherapy targeting 4-1BB agonist therapy.

4-1BB (CD137; TNFRSF9) was initially discovered on T cells (Kwon B S, Weissman S M. cDNA sequences of two inducible T-cell genes. Proc Natl Acad Sci U S A. 1989 86(6):1963-7) and is known to be an activation induced molecule (Choi Y, et al. J Immunother Cancer. 2020 8(2)). In vitro, effects include induction of cell cycle progression, cytokine induction, prevention of activation induced immune cell death (39), and involvement in reshaping T cell metabolism (Menk A V, et al. J Exp Med. 2018 215(4):1091-100; Teijeira A, et al. Cancer Immunol Res. 2018 6(7):798-811). In vivo, 4-1BB activation has been demonstrated as an important part of CD8 T cell robust anti-tumor immune response (Melero I, et al. Nat Med. 1997 3(6):682-5; Buchan S L, et al. Immunity. 2018 49(5):958-70 e7). Indeed, many of the most successful chimeric antigen receptor (CAR) T cells integrate a constitutively active 4-1BB stimulatory domain (Finney HM, et al. J Immunol. 2004 172(1):104-13; Milone M C, et al. Mol Ther. 2009 17(8):1453 -64). Most monoclonal antibodies directed against 4-1BB are hypothesized to act on CD8 T cells to maximize their activation and anti-tumor immune response (Eskiocak U, et al. JCI Insight. 2020 5(5); Innamarato P, et al. J Immunol. 2020 205(10):2893-904). Others have also demonstrated the importance of 4-1BB expression on other immune cells including dendritic cells (Futagawa T, et al. Int Immunol. 2002 14(3):275-86; Zhang B, et al. J Immunol. 2010 184(9):4770-8) and B cells (Zhang X, et al. J Immunol. 2010 184(2):787-95; Schwarz H, et al. Blood. 1995 85(4):1043-52). Besides their role in producing antibodies, B cells are important for priming CD8 (Biswas S, et al. Nature. 2021 591(7850):464-70) and CD4 (Whitmire J K, Asano M S, Kaech S M, Sarkar S, Hannum L G, Shlomchik M J, Ahmed R. Requirement of B cells for generating CD4+ T cell memory. J Immunol. 2009 182(4):1868-76) T cell responses, and playing a critical role in organization of tertiary lymphoid structures, which have been associated with improved outcomes (Cabrita R, et al. Nature. 2020 577(7791):561-5; Helmink B A, et al. Nature. 2020 577(7791):549-55).

This study reports that combining anti-4-1BB immunotherapy with ablative RT allows for vigorous immune activation with recruitment of B cells and CD4 T cells for improved local and distant tumor control. Also demonstrated is the impact of RT on changes in CD4 T cells and 4-1BB expression in human extensive stage small cell lung cancer (ES-SCLC).

### Materials and Methods

### Mice

Age matched C57BL/6J mice from The Jackson Laboratory were maintained by the Moffitt Cancer Center animal facility according to the Association for Assessment and Accreditation of Laboratory Animal Care and National Institute of Health (NIH) standards. All experiments were conducted according to protocols approved by the Institutional Animal Care and Use Committee (IACUC) at the University of South Florida. B16-F10 (ATCC: CRL-6475) melanoma cells transduced with the pAc-neo-ova antigen (Addgene 22533) (B16-ova) were cultured in RPMI supplemented with 0.8 mg/mL G418 and 1% L-glutamine at 37° C. and 5% CO₂. One million B16-ova were then implanted subcutaneously in the bilateral flanks of C57BL/6J mice. Mice that received RT were anesthetized and treated with a small cabinet irradiator (XRAD 320, Precision Xray, North Branford, CT). Three uniform daily fractions of 8 Gy were delivered to the right flank on consecutive days beginning when the tumors reached an average size of 20 mm³, while the remainder of the body was shielded. Antibodies were administered intraperitoneally as described below. LLC murine cells (ATCC, Manassas, VA) were maintained in DMEM, supplemented with 10% FCS and 1% L-glutamine at 37° C. and 5% CO₂. One million LLC cells were implanted subcutaneously in the right flank of C57BL/6J mice and allowed to grow approximately 12 days until the tumor was palpable (local tumor). For those receiving a contralateral tumor, 5×10⁵ LLC cells were injected into the left flank (distant tumor) following the 12-day growth of the right flank tumor. Flank tumors were measured with calipers and tumor volumes calculated as (length×width²)/2.

### Human Specimens

Human peripheral blood from patients with ES-SCLC receiving thoracic RT was collected under a protocol approved by the Institutional Review Board at H. Lee Moffitt Cancer Center (NCT03043599) within 72 hours prior to RT and within 72 hours after the final RT fraction (Perez BA, et al. Int J Radiat Oncol Biol Phys. 2021 109(2):425-35). Informed consent was obtained from all subjects.

### Antibodies

Antibodies targeting 4-1BB were obtained from Compass Therapeutics (mAb22) and BioXCell (3H3, Cat. #BE0239). mAb22 is an anti-mouse 4-1BB antibody. Anti-4-1BB (mAb22) therapy was administered on consecutive days, with 3 total doses of 100 μg delivered concurrently with RT. For immune cell depletion, anti-CD4 (GK1.5, Cat. #BE0003-1, 200 μg), anti-CD8 (Lyt 3.2, Cat. #BE0223, 200 μg), or anti-B220 (RA3.3A1/6.1, Cat. #BE0067, 300 μg) were administered intraperitoneally on day 2 after initiation of RT and anti-4-1BB.

### Flow Cytometry

Tumor, spleen, and draining bilateral lymph nodes were harvested from mice either 5 or 10 days after tumor implantation. Samples were mechanically disassociated, followed by red blood cell lysis prior to staining. Mouse staining was done using antibodies against CD45 (Biolegend, 103113), CD3 (BD Biosciences, 563565), CD4 (BD Biosciences, 612900), CD8 (BioLegend, 612759), CD44 (BioLegend, 103026), B220 (BD Biosciences, 612839), CD80 (Biolegend 104711), CD86 (Biolegend, 105035), CD11c (Biolegend, 117320), CD40L (BD Biosciences 561719), CD103 (Biolegend, 121429), 4-1BB (Biolegend, 106110), CD69 (BD Biosciences 562920). Data were collected using a BD LSRII flow cytometer and gated by lymphocytes, single cells, CD45+ cells, with concomitant B220+ cells, CD3+ cells (either CD4 or CD8) or CD11c+ cells. Human peripheral blood immune cell staining was performed using antibodies against CD3 (BD Biosciences 564809), CD4 (BD Biosciences 564305), CD25 (BD Biosciences 560503, 41BB (BD Biosciences 564091), CD69 (BD Biosciences 557745), CD45RA (BD Biosciences 555488).

### Statistical Analyses

A Kaplan-Meier estimate was performed to measure delay of unirradiated tumor growth by measuring the time from inoculation until the tumor reached a volume of 150 mm³. Tumor growth was censored on the last observation day for mice not reaching volumes of 150 mm³. A log-rank test was conducted to test the difference between treatment groups. Error bars represent the standard error of the mean. All statistical analyses were done using GraphPad Prism 9.0 (GraphPad Prism, RRID: SCR_002798). For flow cytometry, data analysis was done using 2-way ANOVA with Dunnett's multiple comparison correction. p<0.05 was considered statistically significant.

### Results

### B Cells and CD4 T Cells Accumulate in the Tumor Microenvironment After Radiotherapy

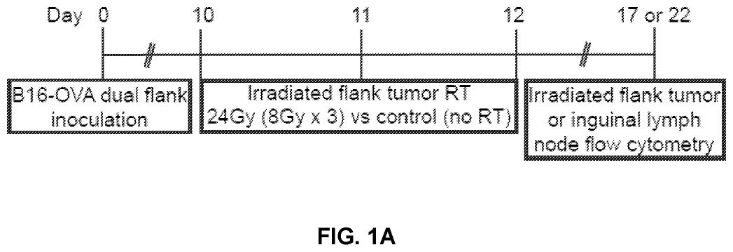

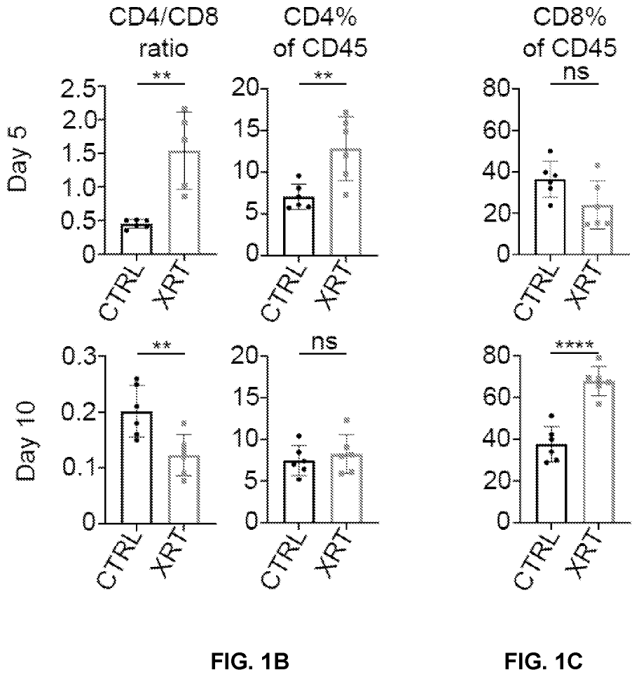

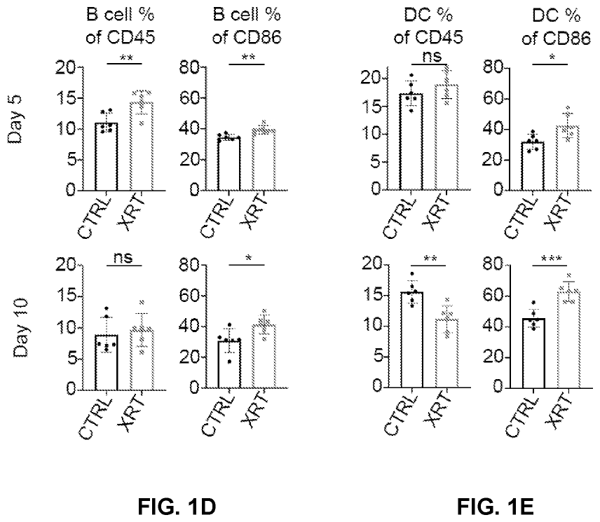

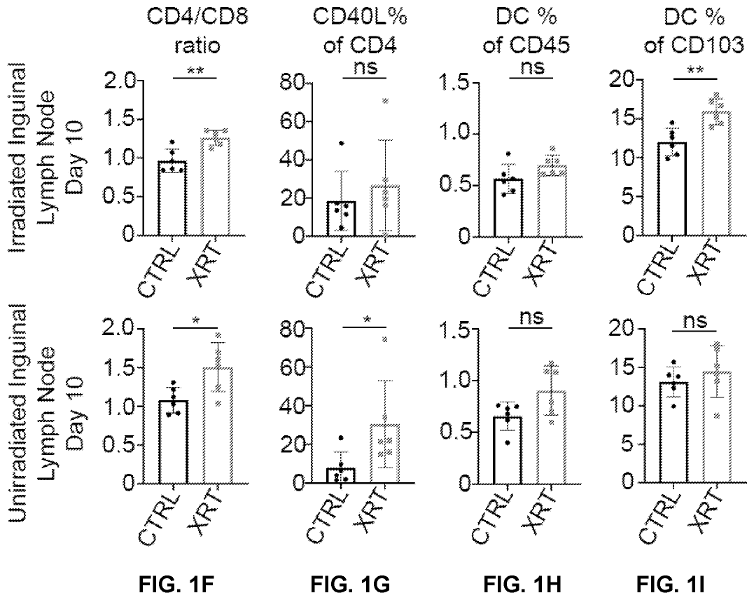

To understand changes in the immune-environment of established tumors in response to RT, C57BL/6J mice were first treated with established bilateral B16-ova flank tumors. At 5 days after initiation of radiotherapy, there was an early influx of CD4 T cells (FIG. 1B), followed by late accumulation of CD8 T cells at ˜Day 10 (FIG. 1C). Coinciding with CD4 T cell accumulation, there was an increase in the number of B cells. These B cells were activated, as indicated by increased CD86 expression from day 5 to day 10 after RT (FIG. 1D). Although no change was detected in the ratio of DCs as a percent of CD45 on day 5 and a decrease in proportion of DCs on day 10 (likely due to significant influx of CD8+ cells), there was an increase in activation of DCs, as evidenced by increased CD86 surface expression at both temporal points (FIG. 1E). At early temporal points, we also found a sharp increase in the CD4/CD8 ratio in the bilateral draining lymph nodes (FIG. 1F), concomitant to an increase in CD40L expression on CD4+cells in the unirradiated draining lymph node (FIG. 1G), in tumor-bearing mice receiving RT, compared to no treatment controls. Though there were no significant changes in the proportion of DCs in bilateral draining lymph nodes (FIG. 1H), CD103⁺ dendritic cells were identified (FIGS. 11), which are increased with radiation treatment in the draining lymph node on the irradiated side, as has been previously reported (Sharabi A B, et al. Cancer Immunol Res. 2015 3(4):345-55).

### Radiotherapy Increases Hallmarks of Antigen Exposure and 4-1BB Expression in T and B Cells in Mice and Humans

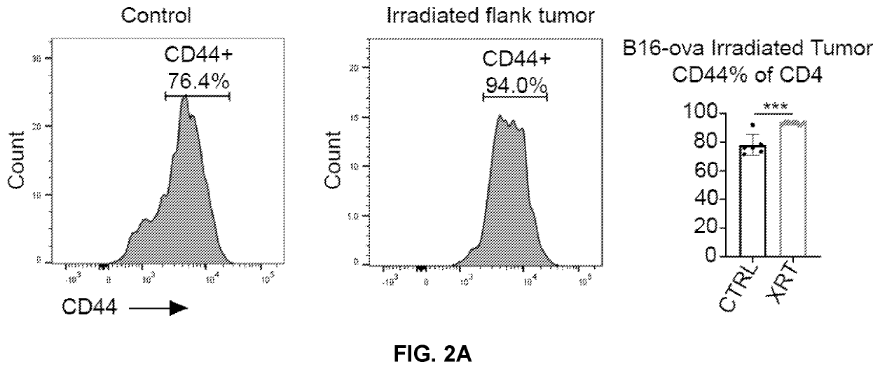

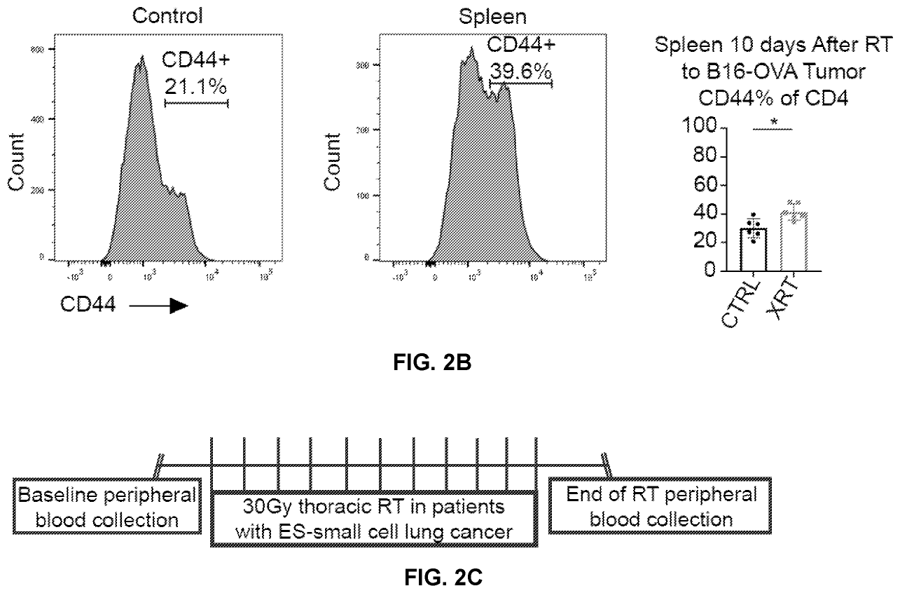

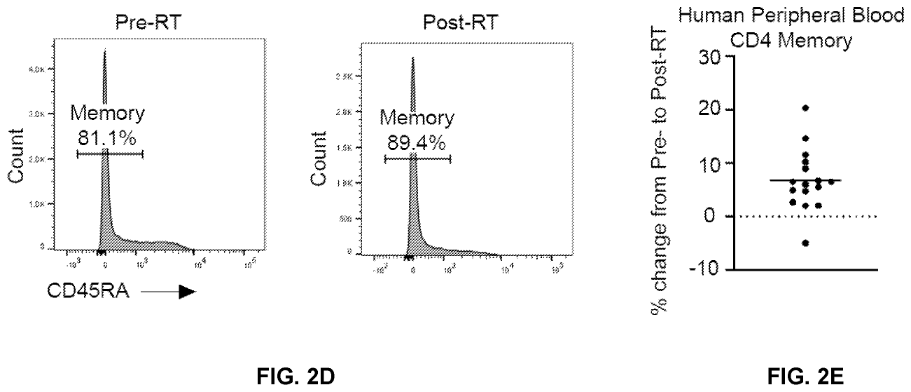

To further study the role of CD4 T cells, we found that after RT, CD4 T cells demonstrate significantly increased markers of antigen exposure, including CD44 and CD45RA, at both the irradiated tumor site (FIG. 2A), and in the spleen (FIG. 2B). The relevance of concurrent CD4 T cell activation was supported by the results of a clinical trial of patients with ES-SCLC receiving 30Gy in 10 fractions of thoracic radiation (FIG. 2C), which also demonstrated decreased expression of CD45RA on in response to RT (FIG. 2D). Additionally, RT resulted in a significant increase in markers of memory differentiation in CD4 T cells (FIG. 2E), while the exhaustion/activation marker PD1 did not show significant change with RT at our analysis time points.

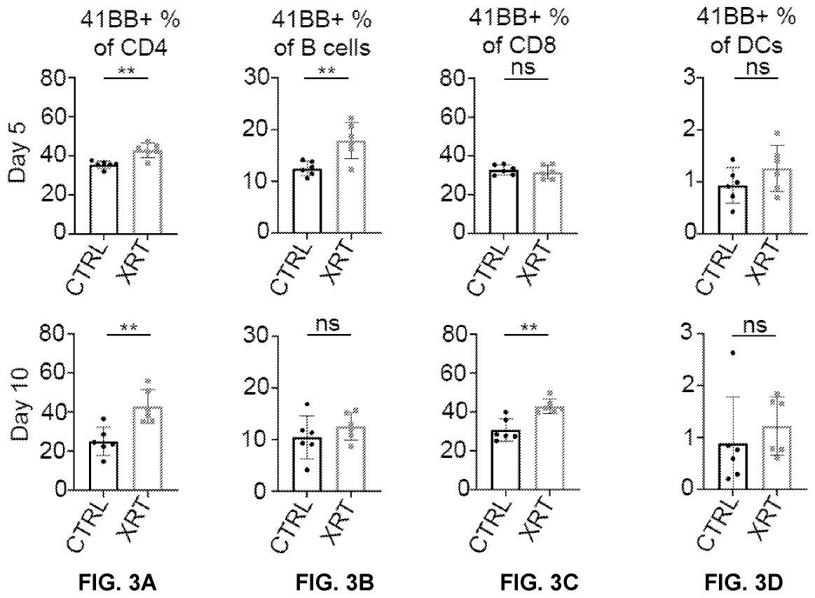

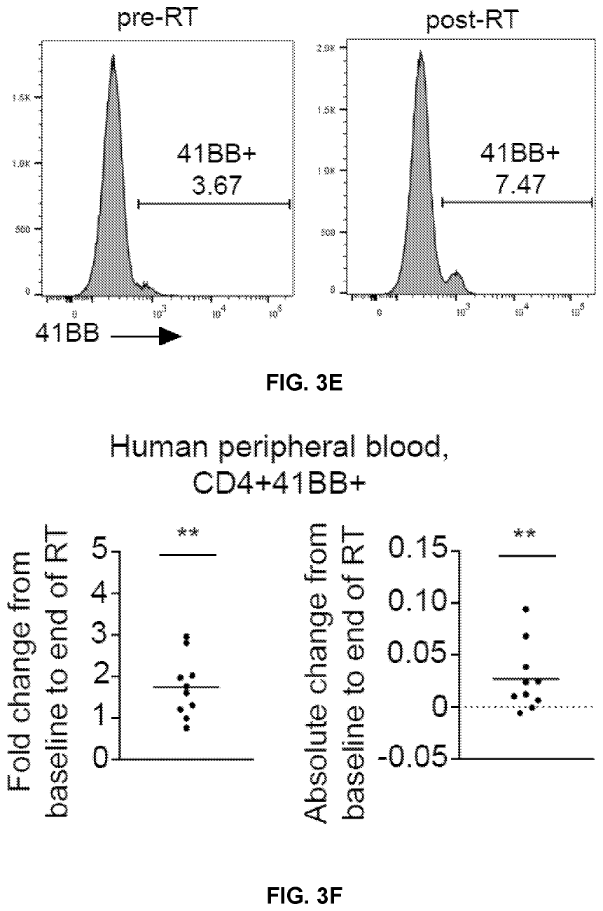

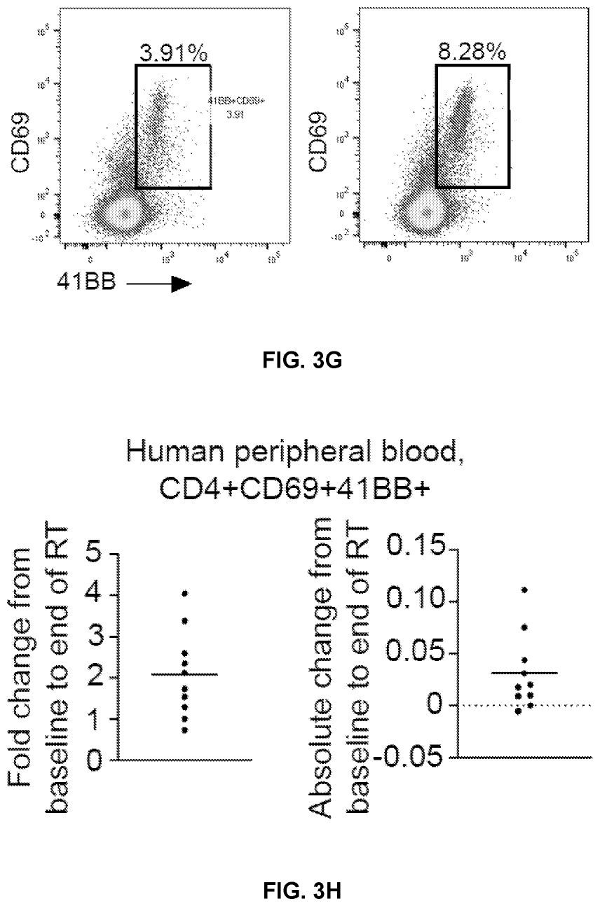

Analysis of additional activation markers in T cells, B cells, and dendritic cells at irradiated tumor sites in tumor-bearing mice showed that a significant percentage of both CD4 T cells (FIG. 3A) and B cells (FIG. 3B) turn on 4-1BB after RT at early time points. Further supporting that CD4 T cell activation precedes the immunostimulatory effects of RT on CD8 T cells, the percentage of 4-1BB positive CD8 lymphocytes only increased after 10 days (FIG. 3C), coinciding with the accumulation of this population at tumor beds (FIG. 1C). There was also a trend toward increase in 4-1BB positive DCs (FIG. 3D), albeit without statistical significance, given the scarcity of this population. Further supporting the clinical relevance of these findings, 4-1BB expression following RT was also significantly increased on peripheral CD4 T cells following thoracic

RT in humans with ES-SCLC (FIGS. 3E-3F), with significant increases in the mobilization of activated 4-1BB⁺ CD4 cells post-RT (FIGS. 3G-3H). Importantly, these peripheral CD4+ cells with increased 4-1BB expression do not upregulate CD25⁺, and are therefore unlikely to correspond to Treg cells. These data suggest that the immunostimulatory effects of RT in fact depend on early activation of CD4 T cells and B cells, with subsequent activation of CD8 T cells at later temporal points. Further, upregulation of 4-1BB on these populations provides a rationale for combined 4-1BB agonists.

### Combining Radiation and Anti-4-1BB Agonists Enhances CD8 T Cell Infiltration and Elicits Abscopal Effects on Non-Irradiated Tumor Sites

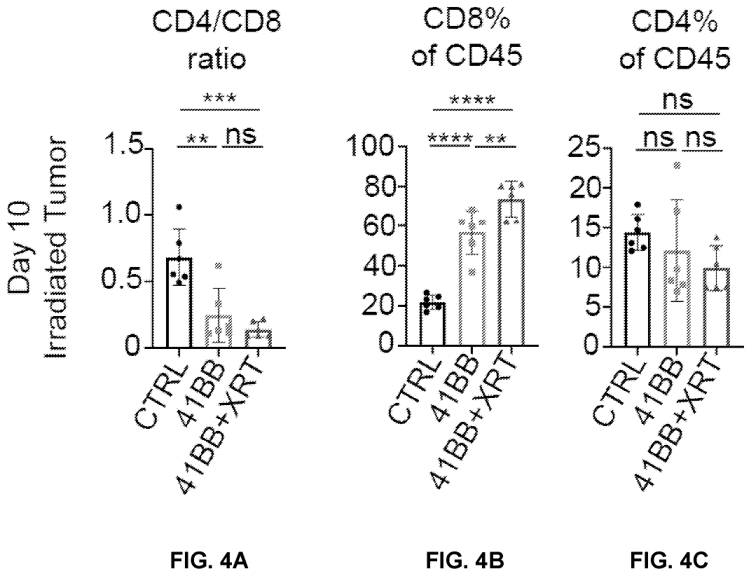

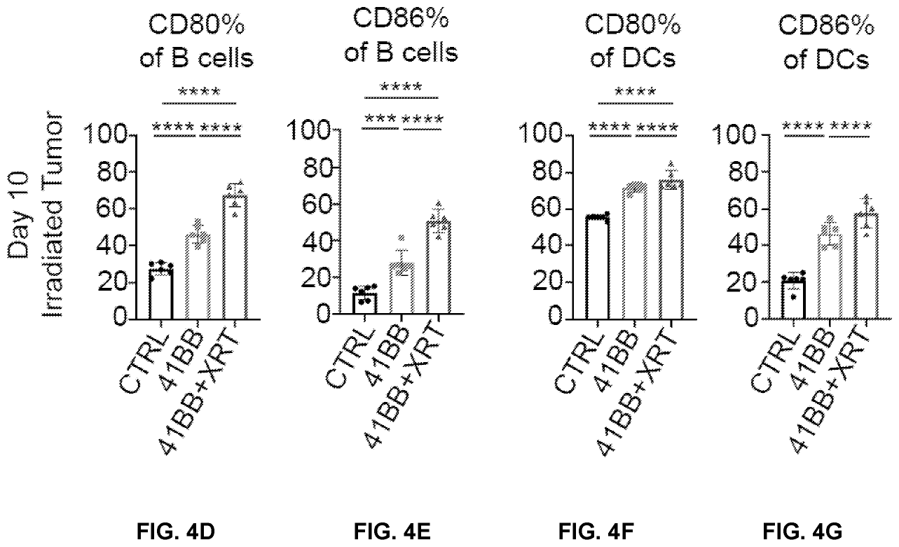

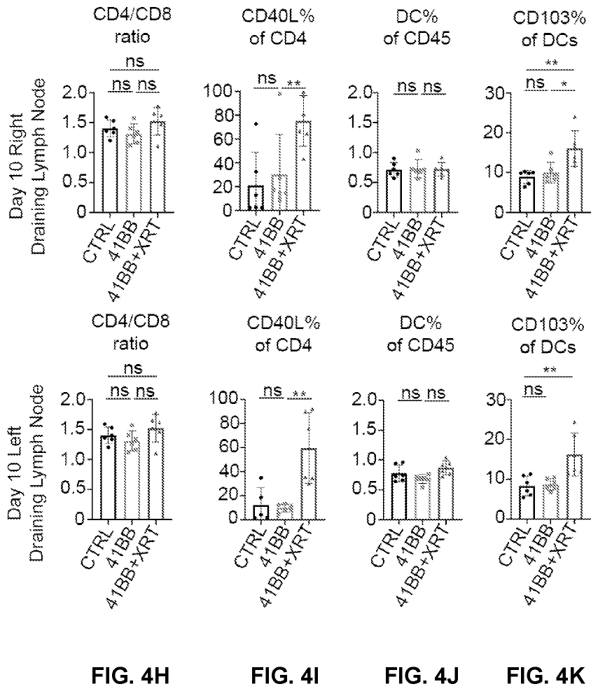

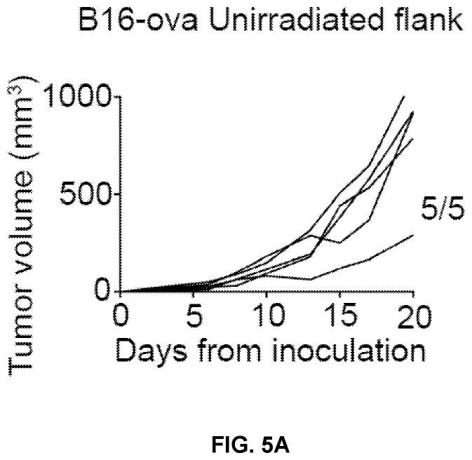

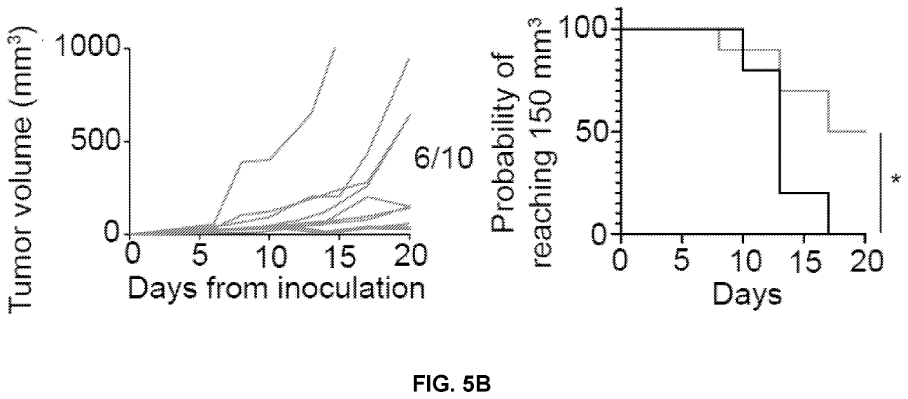

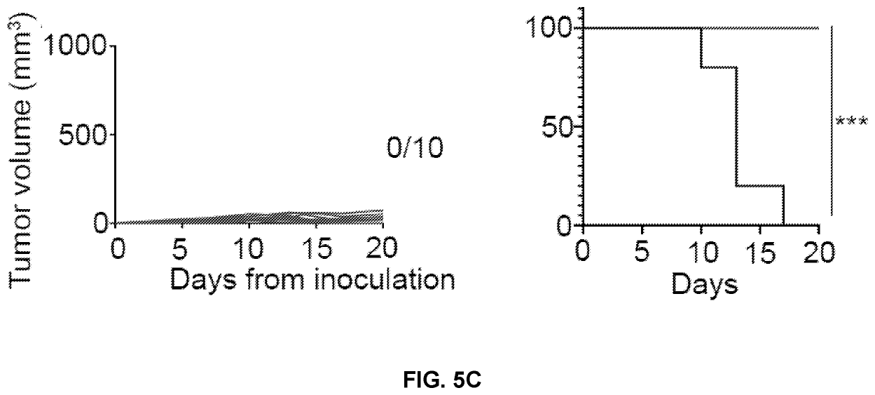

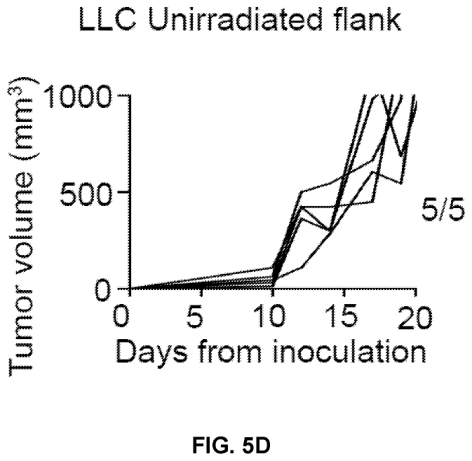

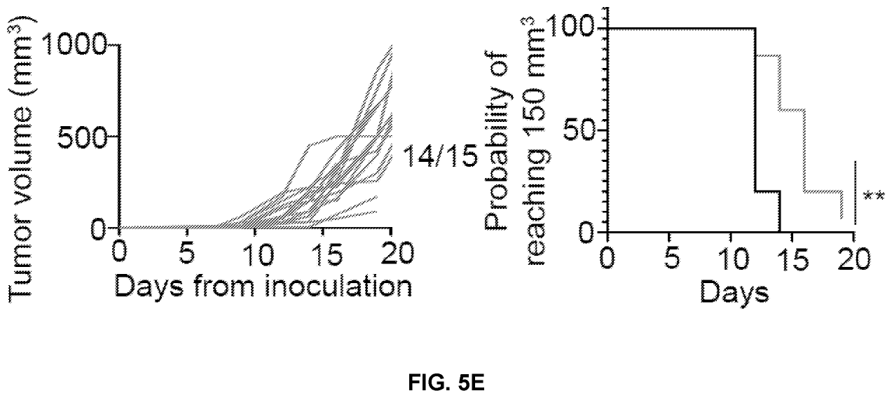

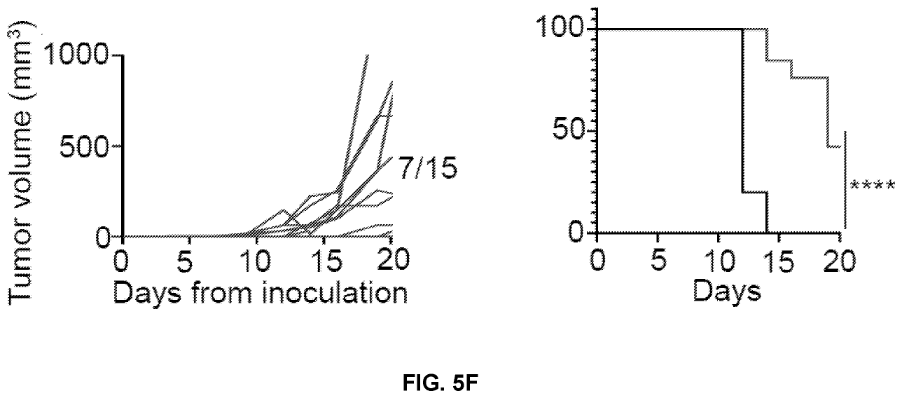

Based on results that showed increased 4-1BB expression with RT, ablative radiation therapy was combined with a 4-1BB agonist, namely mAb22. The 4-1BB antibody is an IgG4 antibody, which demonstrates intermediate characteristics of agonistic activity and works to facilitate FC gamma cross linking, allowing for natural 4-1BB-L/4-1BB interactions to drive T cell activation (Eskiocak U, et al. JCI Insight. 2020 5(5)). Combining RT and 4-1BB agonist (mAb22) resulted in significant increases in the influx of CD8 T cells (FIGS. 4A-4B), compared to no treatment or 4-1BB agonist (mAb22) alone, without significant changes in the CD4 T cell compartment at later temporal points (FIG. 4C). Combination 4-1BB agonist (mAb22) and RT also induced increased proportions of CD80⁺ and CD86⁺ B cells (FIG. 4D-4E) as well as CD80⁺ and CD86⁺ DCs (FIG. 4F-4G). Furthermore, upon evaluation of the draining lymph nodes of both tumor beds while no differences in the proportion of CD4/CD8 T cells were observed (FIG. 4H), combining RT plus 4-1BB agonists resulted in increased surface expression of CD40L among CD4⁺ T cells in tumor-draining lymph nodes (FIG. 41). Though there were no differences in proportion of dendritic cells in the bilateral draining lymph nodes with the combination therapy (FIG. 4J), there was increased accumulation of CD103⁺ dendritic cells (FIG. 4J). Most importantly, utilizing the B16-OVA model the growth of unirradiated tumors was delayed but could not be routinely controlled using RT alone (FIG. 5B) and complete tumor control unirradiated tumor sites could be only achieved using combination therapy (FIG. 5C). To demonstrate the general applicability of these results in a less immunogenic tumor model, we have also evaluated the impact of 41BB agonist (3H3) and RT in a separate Lewis lung carcinoma (LLC) model. Utilizing a similar dual flank approach with RT to the right flank only, RT with anti-41bb agonist significantly improved tumor control at the unirradiated flank compared to RT alone (FIG. 5D-5F). Therefore, in two model systems, RT and 4-1BB agonists elicit abscopal effects that abrogate the growth of tumor lesions distal to irradiated sites, which is associated with distinct changes in the tumor immuno-environment.

### Both B Cells and CD4 T Cells Are Required for the Distinct Therapeutic Benefits of Combined RT and 4-1BB Therapy

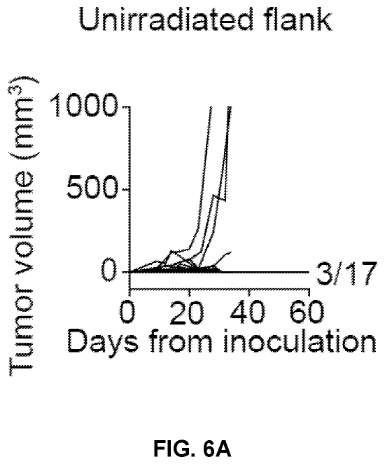

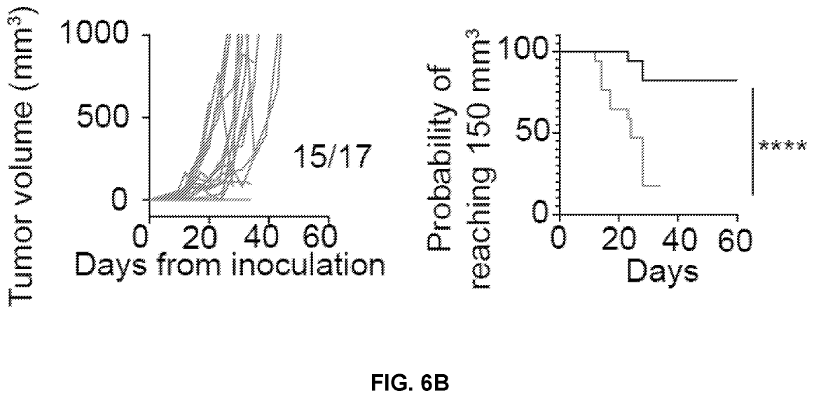

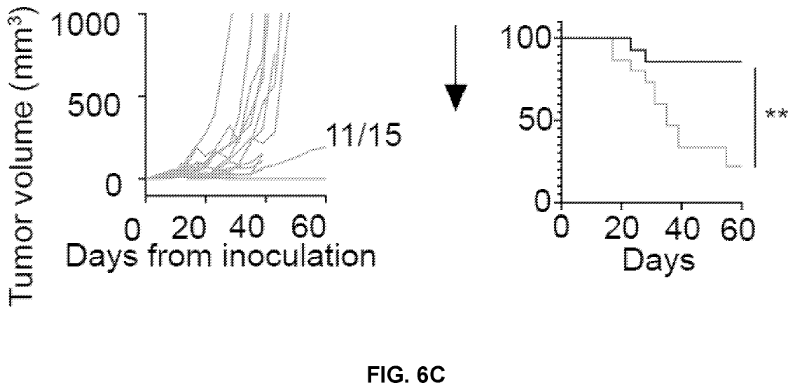

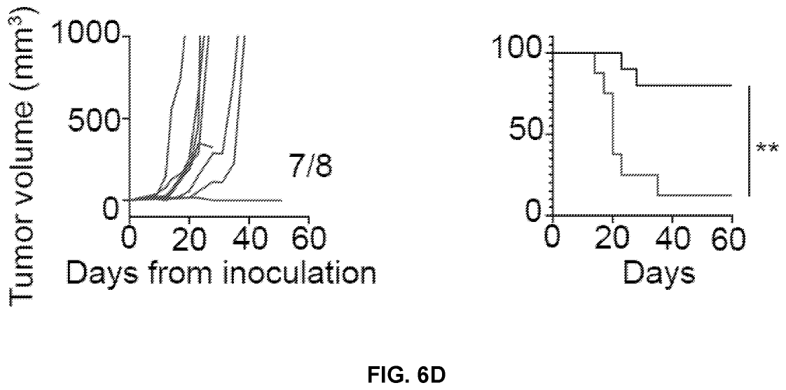

Early accumulation and activation of B cells and CD4 T cells suggest that their crosstalk could be required for CD8 T cell-dependent protective effects elicited by RT plus 4-1BB agonists. Accordingly, it was found that early B cell depletion prevents the control of tumor growth at the unirradiated flank tumor elicited by combined RT and 4-1BB agonist (mAb22), with ˜90% of tumors demonstrating accelerated malignant progression and reaching endpoint significantly sooner than the control cohort (FIG. 6B). Similarly, early depletion of CD4 T cells after combined therapy also allowed the growth of unirradiated tumor sites, with over 70% of tumors demonstrating accelerated growth and reaching endpoint sooner than the control group (FIG. 6C). As expected, depletion of CD8 T cells also resulted in accelerated growth in nearly all tumors, and those mice with CD8 depletion were significantly more likely to reach endpoint sooner than the control group (FIG. 6D). Therefore, the combined effects of RT and 4-1BB agonists in anti-tumor immunity depend on the crosstalk between multiple immune cell populations, whereby the early activation of CD4 T cells and B cells precedes the activation and accumulation of CD8 T cells, which elicit systemic protective anti-tumor immunity beyond irradiated tumor sites.

### Discussion

Radiotherapy and 41BB agonist therapy work synergistically to induce B cell-dependent anti-tumor immune activation and improve tumor control in advanced metastatic melanoma and lung cancer. There was increased B cell activation and migration to the tumor microenvironment in response to RT. The activation and migration of CD8 T cells is even more pronounced with the combination of RT and 4-1BB agonist (mAb22) anti-41BB therapy. In a murine model there was improved delayed malignant progression with the combination therapy, which was eliminated when B cells were depleted. This data strongly suggests that B cells play an important role in response to therapy with 4-1BB agonist (mAb22) and RT.

Also demonstrated was early (day 5) influx of CD4 cells in response to RT, with even further increase with the addition of anti-41BB to the RT. There was also a massive influx of CD8 T cells to the tumor microenvironment at the later temporal point (day 10), both of which are crucial to the control of malignant progression with combination therapy, as demonstrated by CD4 and CD8 T cell depletions. Although not wishing to be bound by theory, the activation and upregulation of 4-1BB, CD40L and interactions with B cells may drive robust immune activation to ultimately stimulate proliferation and activation of CD8 cytotoxic T cell killing.

CD4 T and B cells migrate to the tumor and draining lymph nodes and demonstrate features of activation and proliferation. These effects are increased by the addition of anti-4-1BB therapy, utilizing 4-1BB agonist (mAb22). CD4 T cells have long been minimized because they are not typically involved in direct cytotoxic tumor cell killing. Similarly, B cells have been largely forgotten with so much focus on improving and maximizing CD8 T cell function. The results from this study highlight opportunities for synergy with 4-1bb agonist and RT to act as an in-situ vaccine.

In human samples, there was evidence of increased 41BB expression with increased CD69 in CD4 T cells, which is significantly associated with radiation therapy.

These cells may be coordinating combined humoral and cell mediated immune response in conjunction with B cells and other important immune contributors. Peripherally, no changes in 41BB on B cells or CD8 T cells was detected at the time points evaluated. Data evaluating the draining lymph nodes of irradiated tumors also indicates changes in CD4 cells but not B cells or CD8 T cells at regional (draining lymph nodes) and peripheral sites (spleen). This suggests that following RT CD4 T cells are activated and spreading anti-tumor response.

One reason the CD4 depletion cohort has intermediate/less obvious phenotype compared to B cells and CD8 may be because of Treg depletion which is concurrent. Distant tumor control with RT and 4-1BB agonist (mAb22) depends on B cells and CD4 T cells which play important early roles in RT response. These relevant and activated lymphocytes demonstrate increased surface expression of 41BB after RT. Combined treatment with 4-1BB agonist (mAb22) and RT improves radiation response and systemic anti-tumor response. These results demonstrate that radiation therapy can be successfully combined with systemic immunotherapy, including 4-1BB agonist (mAb22) and future clinical trials to combine these therapies may improve outcomes for patients with melanoma and lung cancer.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating a tumor in a subject comprising
a) treating the tumor with an effective amount of a radiotherapy;
b) treating the subject with an effective amount of a B cell agonist; and
c) administering to the subject an effective amount of a 4-1BB agonist.

2. The method of claim 1, wherein the 4-1BB agonist is an antibody, or antigen binding portion thereof, that specifically binds human CD137.

3. The method of claim 1, wherein the B cell agonist is selected from the group consisting of CpG DNA, IL-2, IL-10, IL-15, IL-6, IFNα, and anti-CD40L.

4. The method of claim 1, wherein step b) is performed about 0 to 5 days after step a).

5. The method of claim 1, wherein step c) is performed about 0 to 5 days after step b).

6. The method of claim 1, further comprising administering to the subject a checkpoint inhibitor.

7. The method of claim 6, wherein the checkpoint inhibitor comprises an anti-PD-1 antibody, anti-PD-L1 antibody, anti-CTLA-4 antibody, or a combination thereof.

8. The method of claim 1, wherein the effective amount of radiation comprises 1-20 Gy per dose fraction.

9. The method of claim 8, wherein the effective amount of radiation comprises 1 to 5 dose fractions.


SEQUENCE LISTING (48 sequences)

SEQ ID NO: 1 - protein, 9 aa, synthetic construct
FTFSSYAMS

SEQ ID NO: 2 - protein, 12 aa, synthetic construct
SAISGSGGSTYY

SEQ ID NO: 3 - protein, 18 aa, synthetic construct
AKDSPFLLDDYYYYYYMD

SEQ ID NO: 4 - protein, 12 aa, synthetic construct
RASQGISSWLAW

SEQ ID NO: 5 - protein, 8 aa, synthetic construct
YAASSLQS

SEQ ID NO: 6 - protein, 10 aa, synthetic construct
QQGHLFPITF

SEQ ID NO: 7 - protein, 12 aa, synthetic construct
RASQNIHNWLAW

SEQ ID NO: 8 - protein, 8 aa, synthetic construct
YKASGLES

SEQ ID NO: 9 - protein, 10 aa, synthetic construct
QQGDRFPLTF

SEQ ID NO: 10 - protein, 12 aa, synthetic construct
RASQSISRWLAW

SEQ ID NO: 11 - protein, 8 aa, synthetic construct
FKASALES

SEQ ID NO: 12 - protein, 10 aa, synthetic construct
QQGNSFPLTF

SEQ ID NO: 13 - protein, 12 aa, synthetic construct
RASQNIDIWLAW

SEQ ID NO: 14 - protein, 8 aa, synthetic construct
YKASGLET

SEQ ID NO: 15 - protein, 10 aa, synthetic construct
QQGNQFPLTF

SEQ ID NO: 16 - protein, 12 aa, synthetic construct
RASQSIGRWLAW

SEQ ID NO: 17 - protein, 8 aa, synthetic construct
FKASALEV

SEQ ID NO: 18 - protein, 12 aa, synthetic construct
RASQSISSWLAW

SEQ ID NO: 19 - protein, 8 aa, synthetic construct
YAASALQS

SEQ ID NO: 20 - protein, 10 aa, synthetic construct
QQGDSFPLTF

SEQ ID NO: 21 - protein, 12 aa, synthetic construct
RASQSINTWLAW

SEQ ID NO: 22 - protein, 8 aa, synthetic construct
YKASALEN

SEQ ID NO: 23 - protein, 8 aa, synthetic construct
YKASALES

SEQ ID NO: 24 - protein, 10 aa, synthetic construct
QQGHSFPLTF

SEQ ID NO: 25 - protein, 12 aa, synthetic construct
RASQSISDWLAW

SEQ ID NO: 26 - protein, 8 aa, synthetic construct
FKASALEG

SEQ ID NO: 27 - protein, 10 aa, synthetic construct
QQGNSFPITF

SEQ ID NO: 28 - protein, 12 aa, synthetic construct
RASQSVDRWLAW

SEQ ID NO: 29 - protein, 8 aa, synthetic construct
YEASALQG

SEQ ID NO: 30 - protein, 10 aa, synthetic construct
QQGDSFPLTF

SEQ ID NO: 31 - protein, 8 aa, synthetic construct
YAASGLQN

SEQ ID NO: 32 - protein, 9 aa, synthetic construct
FTFNYYAMS

SEQ ID NO: 33 - protein, 12 aa, synthetic construct
SAIDGSGDNTTY

SEQ ID NO: 34 - protein, 12 aa, synthetic construct
AAISGSGDGTYY

SEQ ID NO: 35 - protein, 12 aa, synthetic construct
AAISGGGDATYY

SEQ ID NO: 36 - protein, 9 aa, synthetic construct
FTFYGYAMS

SEQ ID NO: 37 - protein, 12 aa, synthetic construct
SSISGSGDVTYY

SEQ ID NO: 38 - protein, 9 aa, synthetic construct
FTFRNYAMS

SEQ ID NO: 39 - protein, 12 aa, synthetic construct
SAISGFGESTYY

SEQ ID NO: 40 - protein, 9 aa, synthetic construct
FTFNYYAMN

SEQ ID NO: 41 - protein, 12 aa, synthetic construct
AAISGSGGRTYY

SEQ ID NO: 42 - protein, 12 aa, synthetic construct
AAISGSGDSTYY

SEQ ID NO: 43 - protein, 12 aa, synthetic construct
SAISGSGDTTYY

SEQ ID NO: 44 - protein, 9 aa, synthetic construct
FTFGWYAMS

SEQ ID NO: 45 - protein, 12 aa, synthetic construct
RASQDIGDWLAW

SEQ ID NO: 46 - protein, 8 aa, synthetic construct
YKASGLQS

SEQ ID NO: 47 - protein, 12 aa, synthetic construct
SAISGSGDSTYY

SEQ ID NO: 48 - protein, 12 aa, synthetic construct
SAISGSGGNTSY